SEQUENCE LISTING (1 sequence)

SEQ ID NO: 1 - protein, 6 aa, Homo sapiens
LKKTET

(19) United States
(12) Patent Application Publication
(10) Pub. No.: US 2011/0020449 A1
(43) Pub. Date: Jan. 27, 2011

(54) METHODS OF TREATING DISORDERS OF THE EYE AND SURROUNDING TISSUE WITH THYMOSIN BETA 4 (TB4), ANALOGUES, ISOFORMS AND OTHER DERIVATIVES

(75) Inventors: ALLAN L. GOLDSTEIN, WASHINGTON, DC (US); GABRIEL SOSNE, OAK PARK, MI (US)

Correspondence Address: ROTHWELL, FIGG, ERNST & MANBECK, P.C., 1425 K STREET, N.W., SUITE 800, WASHINGTON, DC, 20005, US

(73) Assignee: REGENERX BIOPHARMACEUTICALS, INC., ROCKVILLE, MD (US)

(21) Appl. No.: 12/775,959

(22) Filed: May 7, 2010

Related U.S. Application Data
(63) Continuation-in-part of application No. 11/841,575, filed on Aug. 20, 2007.
Application No. 11/841,575 is a continuation-in-part of application No. 10/471,621, filed on Feb. 26, 2004, now abandoned.
Application No. 10/471,621 is a continuation-in-part of application No. 09/772,445, filed on Jan. 29, 2001.
Application No. 09/772,445 is a continuation of application No. PCT/US99/17282, filed on Jul. 29, 1999.
(60) Provisional application No. 60/275,645, filed on Mar. 15, 2001.
(60) Provisional application No. 60/094,690, filed on Jul. 30, 1998.

(51) Int. Cl. A61K 38/00; A61K 9/00; A61P 27/02

(52) U.S. Cl. 424/484; 514/20.8

(57) ABSTRACT

Pain or irritation of the eyes, caused by injury due to dry eye syndrome, chemical burns or the like can be accompanied by corneal stromal edema. It has been discovered that administration of thymosin β4 and/or oxidized thymosin β4 to cornea in need of treatment of corneal stromal edema is a useful treatment for decreasing such corneal stromal edema.

### CROSS-REFERENCE TO RELATED APPLICATION

This application is a continuation-in-part of U.S. application Ser. No. 11/841,575, filed Aug. 20, 2007, which is a continuation-in-part of U.S. application Ser. No. 10/471,621, filed Feb. 26, 2004, which is a §371 of PCT/US02/07730, filed Mar. 14, 2002, which claims the benefit of U.S. Provisional Application No. 60/275,645, filed Mar. 15, 2001. The present application also is a continuation-in-part of U.S. application Ser. No. 09/772,445, filed Jan. 29, 2001, which is a continuation of PCT/US99/17282, filed Jul. 29, 1999, which claims the benefit of U.S. Provisional Application No. 60/094,690, filed Jul. 30, 1998. The previously mentioned applications are explicitly incorporated herein by reference in their entirety for all purposes.

### BACKGROUND OF THE INVENTION

1. Field of the Invention

The present invention relates to the field of the treatment of eye disorders such as “dry eye syndrome.”

2. Description of the Background Art

The phenomenon called “dry eye syndrome” may occur not only with advancing age due to normal aging of the glands of the eye, but also due to other degenerative changes and environmental factors and can occur at any age. Dry eye syndrome results from deleterious changes in the physiological, biochemical and immunological properties of the eye.

Certain patients experience constant pain from eye irritation caused from the decline of the quality or quantity of tears. Such patients have a sandy or gritty sensation that, if untreated, can lead to scarring or ulceration of the cornea, and thus loss of vision. In many cases, dry eye results from disorders of the various glands which work together to produce normal tears. Tears themselves are a complex combination of substances which form three layers on the eye. The very thin outer layer contains lipids from the Meibomian glands in the eyelid, to reduce evaporation. The lacrimal glands produce the middle watery layer that keeps the salinity and the acidity of the tears at proper levels. This middle layer also carries antibodies and other immune defense agents to defend the eye against infection. The inner mucous layer helps the tear film “stick” to the cornea and stay intact.

There may be many causes of dry eye syndrome. The normal aging of tear glands, as well as specific diseases and disorders, may cause changes in the amount and condition of tears produced. For example, Sjögren's syndrome is an immune system disorder characterized by inflammation and dryness of the mouth, eyes, and other mucous membranes, damages the lacrimal glands, and this damage affects tear production. Decreased sensitivity of the cornea can also lead to insufficient production of tears. This lack of sensitivity can be brought on by a disease known as “neurotrophic keratitis” as well as by some types of contact lens wear. Excessive evaporation of tears can also cause dry eye syndrome. Such evaporation may be caused by “meibomitis,” which results from infection and inflammation of the meibomian glands in the eyelids. People with unusually large eyes, as well as those who suffer from thyroid disease, may also experience dry eye syndrome caused by excessive evaporation. Dry eye can also result from unusual facial anatomy or irregularities in the cornea, resulting in uneven or inadequate tear coverage of the eye. Some patients suffer from dry eye as a result of medications such as antibiotics, antihistamines, diuretics, and anti-diarrheals, which can dry up the mucous membranes. Hormonal changes, such as may be associated with menopause and the aging process, can also affect secretions of Tβ4 from the tear glands and result in dry eyes and inflammation of the eye.

A number of approaches have been reported to delay and/or to decrease such eye disorders. Dry eyes are typically treated by applying artificial tears and ointments. These give temporary relief, but usually do not arrest or reverse damage to the eye. Eye drops which are aimed at restoring the electrolyte balance of the tears and promoted healing of the cornea are in development. There is also evidence that dry eye may be treated with hormone therapy or antibodies. In addition, some forms of dry eye benefit from the placement of tiny plugs in the ducts that drain tears from the eye. For severe forms of dry eye, special goggles called “moisture-chamber spectacles” can be worn.

There remains a need in the art for improved methods and compositions for the treatment of dry eye disorders.

### SUMMARY OF THE INVENTION

Accordingly, an embodiment of the present invention includes a method of treatment for decreasing corneal stromal edema in a subject in need of such treatment comprising topically administering to a cornea of the subject an effective amount of a composition comprising a pharmaceutically acceptable carrier and a polypeptide comprising at least thymosin β4 (Tβ4) or oxidized Tβ4, said polypeptide being present in said composition at a concentration within a range of about 0.001-10% by weight, so as to decrease corneal stromal edema in said subject.

### DETAILED DESCRIPTION OF THE INVENTION

The present invention is based on a discovery that actin-sequestering peptides such as thymosin β4 (Tβ4) and other actin-sequestering peptides containing amino acid sequence LKKTET (SEQ ID NO:1) or conservative variants thereof, promote reversal of or inhibit eye degeneration such as may be associated with or result from dry eye syndrome. The potential clinical applications might include disorders due to inflammatory conditions e.g., dry eyes, uveitis, iritis, post operative cataract surgery, LASIK or PRK, corneal melts due to rheumatoid arthritis, systemic lupus erythematosus, Mooren's ulcers, Sjögrens syndrome, etc. Other applications could be keratitis due to bacterial, viral, mycobacterial or fungal pathogens. Still other applications could be due to metabolic diseases of the eye such as caused by diabetes (keratopathy and retinopathy) or as a result of chemical injury, trauma and abrasions.

Thymosin β4 was initially identified as a protein that is up regulated during endothelial cell migration and differentiation in vitro. Thymosin β4 was originally isolated from the thymus and is a 43 amino acid, 4.9 kDa ubiquitous polypeptide identified in a variety of tissues. Several roles have been ascribed to this protein including a role in a endothelial cell differentiation and migration, T cell differentiation, actin sequestration and vascularization.

In accordance with one embodiment, the invention is a method of treatment for promoting reversal of or inhibiting dry eye syndrome comprising administering to a subject in need of such treatment an effective amount of a composition comprising an agent that stimulates production of an eye degeneration-inhibiting polypeptide comprising amino acid sequence LKKTET (SEQ ID NO:1), or a conservative variant thereof having eye degeneration-inhibiting activity, preferably Thymosin β4, an isoform of Thymosin β4, oxidized Thymosin β4 or an antagonist of Thymosin β4.

The present invention promotes the healing and reversal of inflammatory, degenerative, immunological and other disorders of the eye and surrounding tissue.

Compositions which may be used in accordance with the present invention include Thymosin β4 (Tβ4), Tβ4 isoforms, oxidized Tβ4, polypeptides comprising the amino acid sequence LKKTET (SEQ ID NO:1) or conservative variants thereof having eye degeneration-inhibiting activity. International Application Serial No. PCT/US99/17282, incorporated herein by reference, discloses isoforms of Tβ4 which may be useful in accordance with the present invention as well as amino acid sequence LKKTET (SEQ ID NO:1) and conservative variants thereof having eye degeneration-inhibiting activity, which may be utilized with the present invention. International Application Serial No. PCT/GB99/00833 (WO 99/49883), incorporated herein by reference, discloses oxidized Thymosin β4 which may be utilized in accordance with the present invention. Although the present invention is described primarily hereinafter with respect to Tβ4 and Tβ4 isoforms, it is to be understood that the following description is intended to be equally applicable to amino acid sequence LKKTET (SEQ ID NO:1), conservative variants thereof having eye degeneration-inhibiting activity, as well as oxidized Thymosin β4.

In one embodiment, the invention provides a method of treatment for promoting reversal of or inhibiting eye degeneration, such as may be associated with dry eye syndrome, comprising administering to a subject in need of such treatment an effective amount of a composition comprising an eye degeneration-inhibiting polypeptide comprising amino acid sequence LKKTET (SEQ ID NO:1), or a conservative variant thereof having eye degeneration-inhibiting activity. The contacting may be topically or systemically. Examples of topical administration include, for example, contacting the eye with a solution, lotion, salve, gel, cream, paste, spray, suspension, dispersion, hydrogel, ointment, or oil comprising Tβ4. Systemic administration includes, for example, intravenous, intraperitoneal, intramuscular injections of a composition containing Tβ4 or a Tβ4 isoform. A subject may be any mammal, preferably human.

A composition in accordance with the present invention can be administered daily, every other day, etc., with a single application or multiple applications per day of administration, such as applications 2, 3, 4 or more times per day of administration.

Tβ4 isoforms have been identified and have about 70%, or about 75%, or about 80% or more homology to the known amino acid sequence of Tβ4. Such isoforms include, for example, Tβ4ala, Tβ9, Tβ10, Tβ11, Tβ12, Tβ13, Tβ14 and Tβ15. Similar to Tβ4, the Tβ10 and Tβ15 isoforms have been shown to sequester actin. Tβ4, Tβ10 and Tβ15, as well as these other isoforms share an amino acid sequence, LKKTET (SEQ ID NO:1), that appears to be involved in mediating actin sequestration or binding. Although not wishing to be bound to any particular theory, the activity of Tβ4 isoforms may be due, in part, to the ability to polymerize actin. For example, Tβ4 can modulate actin polymerization in the eye (e.g. β-thymosins appear to depolymerize F-actin by sequestering free G-actin). Tβ4's ability to modulate actin polymerization may therefore be due to all, or in part, its ability to bind to or sequester actin via the LKKTET (SEQ ID NO:1) sequence. Thus, as with Tβ4, other proteins which bind or sequester actin, or modulate actin polymerization, including Tβ4 isoforms having the amino acid sequence LKKTET (SEQ ID NO:1), are likely to reduce dry eye syndrome, alone or in a combination with Tβ4, as set forth herein.

Thus, it is specifically contemplated that known Tβ4 isoforms, such as Tβ4^{ala}, Tβ9, Tβ10, Tβ11, Tβ12, Tβ13, Tβ14 and Tβ15, as well as Tβ4 isoforms not yet identified, will be useful in the methods of the invention. As such Tβ4 isoforms are useful in the methods of the invention, including the methods practiced in a subject. The invention therefore further provides pharmaceutical compositions comprising Tβ4, as well as Tβ4 isoforms Tβ4^{ala}, Tβ9, Tβ10, Tβ11, Tβ12, Tβ13, Tβ14 and Tβ15, and a pharmaceutically acceptable carrier.

In addition, other proteins having actin sequestering or binding capability, or that can mobilize actin or modulate actin polymerization, as demonstrated in an appropriate sequestering, binding, mobilization or polymerization assay, or identified by the presence of an amino acid sequence that mediates actin binding, such as LKKTET (SEQ ID NO:1), for example, can similarly be employed in the methods of the invention. Such proteins include gelsolin, vitamin D binding protein (DBP), profilin, cofilin, depactin, DNaseI, vilin, fragmin, severin, capping protein, β-actinin, actobindin and acumentin, for example. As such methods include those practiced in a subject, the invention further provides pharmaceutical compositions comprising gelsolin, vitamin D binding protein (DBP), profilin, cofilin, depactin, DNaseI, vilin, fragmin, severin, capping protein, β-actinin, actobindin and acumentin as set forth herein. Thus, the invention includes the use of a dry eye syndrome reducing polypeptide comprising the amino acid sequence LKKTET (SEQ ID NO:1) and conservative variants thereof.

As used herein, the term “conservative variant” or grammatical variations thereof denotes the replacement of an amino acid residue by another, biologically similar residue. Examples of conservative variations include the replacement of a hydrophobic residue such as isoleucine, valine, leucine or methionine for another, the replacement of a polar residue for another, such as the substitution of arginine for lysine, glutamic for aspartic acids, or glutamine for asparagine, and the like.

Tβ4 has been localized to a number of tissue and cell types and thus, agents which stimulate the production of Tβ4 can be added to or comprise a composition to effect Tβ4 production from a tissue and/or a cell. Such agents include members of the family of growth factors, such as insulin-like growth factor (IGF-1), platelet derived growth factor (PDGF), epidermal growth factor (EGF), transforming growth factor beta (TGF-β), basic fibroblast growth factor (bFGF), thymosin α1 (Tα1) and vascular endothelial growth factor (VEGF). More preferably, the agent is transforming growth factor beta (TGF-β) or other members of the TGF-β superfamily. Tβ4 compositions of the invention may reduce dry eye syndrome by effectuating growth of the connective tissue through extracellular matrix deposition, cellular migration and downregulation of inflammatory cytokines.

Additionally, agents that assist or stimulate dry eye syndrome reduction may be added to a composition along with Tβ4 or a Tβ4 isoform. Such agents include angiogenic agents, growth factors, agents that direct differentiation of cells, agents that promote migration of cells and agents that stimulate the provision of extracellular matrix material in the eye. For example, and not by way of limitation, Tβ4 or a Tβ4 isoform alone or in combination can be added in combination with any one or more of the following agents: VEGF, KGF, FGF, PDGF, TGFβ, IGF-1, IGF-2, IL-1, prothymosin α and thymosin α1 in an effective amount.

The invention also includes a pharmaceutical composition comprising a therapeutically effective amount of Tβ4 or a Tβ4 isoform in a pharmaceutically acceptable carrier. Such carriers include those listed above with reference to parenteral administration.

The actual dosage or reagent, formulation or composition that inhibits or promotes reversal of dry eye syndrome may depend on many factors, including the size and health of a subject. However, persons of ordinary skill in the art can use teachings describing the methods and techniques for determining clinical dosages as disclosed in PCT/US99/17282, supra, and the references cited therein, to determine the appropriate dosage to use. Tβ4, or its analogues, isoforms or derivatives, may be administered in any suitable amount which are effective for the treatment of dry eye or similar disorders. For example, Tβ4 may be administered in dosages within the range of about 0.1-50 micrograms of Tβ4, more preferably in amounts of about 1-25 micrograms Tβ4. The Tβ4 may be administered as a one-time treatment, or may be administered daily, twice per day, three times per day, etc., or on alternate days and the like, until the desired results are obtained.

Suitable topical formulations include Tβ4 or a Tβ4 isoform at a concentration within the range of about 0.001-10% by weight, more preferably within the range of about 0.01-0.1% by weight, most preferably about 0.05% by weight.

The therapeutic approaches described herein involve various routes of administration or delivery of reagents or compositions comprising the Tβ4 or other compounds of the invention, including any conventional administration techniques (for example, but not limited to, topical administration, local administration, or systemic administration), to a subject. The methods and compositions using or containing Tβ4 or other compounds of the invention may be formulated into pharmaceutical compositions by admixture with pharmaceutically acceptable non-toxic excipients or carriers.

The invention includes use of antibodies which interact with Tβ4 peptide or functional fragments thereof. Antibodies which consist essentially of pooled monoclonal antibodies with different epitopic specificities, as well as distinct monoclonal antibody preparations are provided. Monoclonal antibodies are made from antigen containing fragments of the protein by methods well known to those skilled in the art as disclosed in PCT/US99/17282, supra. The term antibody as used in this invention is meant to include monoclonal and polyclonal antibodies.

In yet another embodiment, the invention provides a method of treating a subject by administering an effective amount of an agent which modulates Tβ4 gene expression. The term “modulate” refers to inhibition or suppression of Tβ4 expression when Tβ4 is over expressed, and induction of expression when Tβ4 is under expressed. The term “effective amount” means that amount of Tβ4 agent which is effective in modulating Tβ4 gene expression resulting in reducing the symptoms of the Tβ4 associated dry eye syndrome. An agent which modulates Tβ4 or Tβ4 isoform gene expression may be a polynucleotide for example. The polynucleotide may be an antisense, a triplex agent, or a ribozyme. For example, an antisense directed to the structural gene region or to the promoter region of Tβ4 may be utilized.

In another embodiment, the invention provides a method for utilizing compounds that modulate Tβ4 activity. Compounds that affect Tβ4 activity (e.g., antagonists and agonists) include peptides, peptidomimetics, polypeptides, chemical compounds, minerals such as zincs, and biological agents.

While not be bound to any particular theory, it is believed that the present invention may promote reversal of or inhibit eye degeneration associated with dry eye syndrome by inducing terminal deoxynucleotidyl transferase (a non-template directed DNA polymerase), to decrease the levels of one or more inflammatory cytokines, and to act as a chemotactic factor for endothelial cells, and thereby inhibit or promote reversal of degenerative changes in the eyes brought about by aging or other degenerative or environmental factors.

### Example 1

Tears from healthy young people under the age of 40 and older people over the age of 40 were examined for levels of Tβ4. It was found that Tβ4 is present at highest levels in tears of healthy young people, and that Tβ4 in tears decreases significantly with age and menopause. Thus, dry eye syndrome and inflammation of eyes may be due to deficiency of Tβ4 in tears. Therefore, administering Tβ4 may reduce inflammation, promote healing of inflamed eyes and mucosa, and stimulate production of tears via healing of the glands of the eye responsible for tear production.

### Example 2

Disks of Whatman™ filter paper (size 50) were cut with a 2 mm diameter trephine. The disks were soaked in 1.0 N NaOH and applied to the central cornea of isoflourane-anesthetized mice for 30 seconds. The eyes then were irrigated with 10 ml of PBS and subsequently treated with either Tβ4 (5 mg-5 ml) or a similar volume of PBS (as control) topically twice daily for seven days. After seven days, marked differences between the PBS-treated and the Tβ4-treated eyes were noted. The PBS-treated eyes exhibit markedly edematous and inflamed corneas and the anterior chamber contained marked hyphema and an intense inflammatory cell infiltrate. In contrast, the Tβ4-treated corneas showed decreased stromal edema and more regularly arranged stromal lamellae. The overall anatomical integrity of the anterior segment of the Tβ4-treated as compared to PBS-treated eyes was markedly more normal in appearance.

Transmission electron microscopic analysis also was done at day 7 after treatment with PBS and Tβ4. Corneas treated with Tβ4 revealed a more regular alignment of epithelial intercellular junctions and less vacuolization between cell layers. Similarly, the PBS-treated corneas demonstrated a marked inflammatory infiltrate in areas of stromal digestion and edema, whereas the stroma of the Tβ4-treated corneas appeared intact with more regularly spaced collagen lamellae.

1. A method of treatment for decreasing corneal stromal edema in a subject in need of such treatment, comprising topically administering to a cornea of the subject an effective amount of a composition comprising a pharmaceutically acceptable carrier and a polypeptide comprising at least one of thymosin β4 (Tβ4) or oxidized Tβ4, said polypeptide being present in said composition at a concentration within a range of about 0.001-10% by weight, so as to decrease corneal stromal edema in said subject.

2. The method of claim 1 wherein said composition additionally comprises at least one of an isoform of Tβ4, Tβ4^{ala}, Tβ9, Tβ10, Tβ11, Tβ12, Tβ13, Tβ14, Tβ15, gelsolin, vitamin D binding protein (DBP), profilin, cofilin, depactin, DNaseI, vilin, fragmin, severin, capping protein, β-actinin, actobindin or acumentin.

3. The method of claim 1 wherein said polypeptide comprises Thymosin β4 (Tβ4).

4. The method of claim 1 wherein said composition additionally comprises at least one of Tβ4^{ala}, Tβ9, Tβ10, Tβ11, Tβ12, Tβ13, Tβ14 or Tβ15.

5. The method of claim 1 wherein said composition is in a form of a solution, gel, cream, paste, lotion, spray, suspension, dispersion, salve, hydrogel or ointment.

6. The method of claim 1 wherein said polypeptide is recombinant or synthetic.

7. The method of claim 1 wherein said range is about 0.01-0.1% by weight.

8. The method of claim 1 wherein said concentration is about 0.05% by weight.

9. A method of treatment for decreasing corneal stromal edema in a subject in need of such treatment, comprising topically administering to a cornea of the subject an effective amount of a composition comprising a pharmaceutically acceptable carrier and a polypeptide comprising at least one of thymosin β4 (Tβ4) or oxidized Tβ4, said polypeptide being administered to said subject at a dosage within a range of about 0.1-50 micrograms, so as to decrease corneal stromal edema in said subject.

10. The method of claim 9 wherein said composition additionally comprises at least one of an isoform of Tβ4, Tβ4^{ala}, Tβ9, Tβ10, Tβ11, Tβ12, Tβ13, Tβ14, Tβ15, gelsolin, vitamin D binding protein (DBP), profilin, cofilin, depactin, DNaseI, vilin, fragmin, severin, capping protein, β-actinin, actobindin or acumentin.

11. The method of claim 9 wherein said polypeptide comprises Thymosin β4 (Tβ4).

12. The method of claim 9 wherein said composition additionally comprises at least one of Tβ4^{ala}, Tβ9, Tβ10, Tβ11, Tβ12, Tβ13, Tβ14 or Tβ15.

13. The method of claim 9 wherein said composition is in a form of a solution, gel, cream, paste, lotion, spray, suspension, dispersion, salve, hydrogel or ointment.

14. The method of claim 9 wherein said polypeptide is recombinant or synthetic.

15. The method of claim 9 wherein said range is about 1-25 micrograms.

16. A method of treatment for reducing vacuolization between cell layers of a cornea in an eye of a subject in need of such treatment, comprising administering to the subject an effective amount of a composition comprising at least one of thymosin β4 (Tβ4), oxidized Tβ4, so as to reduce vacuolization between cell layers of said cornea.

17. The method of claim 16 wherein said composition additionally comprises at least one of an isoform of Tβ4, Tβ4^{ala}, Tβ9, Tβ10, Tβ11, Tβ12, Tβ13, Tβ14, Tβ15, gelsolin, vitamin D binding protein (DBP), profilin, cofilin, depactin, DNaseI, vilin, fragmin, severin, capping protein, β-actinin, actobindin or acumentin.

18. The method of claim 16 wherein said polypeptide comprises Thymosin β4 (Tβ4).

19. The method of claim 16 wherein said composition additionally comprises at least one of Tβ4^{ala}, Tβ9, Tβ10, Tβ11, Tβ12, Tβ13, Tβ14 or Tβ15.

20. The method of claim 16 wherein said composition is administered systemically.

21. The method of claim 16 wherein said composition is administered topically.

22. The method of claim 16 wherein said composition is in a form of a solution, gel, cream, paste, lotion, spray, suspension, dispersion, salve, hydrogel or ointment.

23. The method of claim 16 wherein said polypeptide is recombinant or synthetic.

24. The method of claim 16 wherein said polypeptide is present in said composition at a concentration within a range of about 0.001-10% by weight.

25. The method of claim 24 wherein said range is about 0.01-0.1% by weight.

26. The method of claim 24 wherein said concentration is about 0.05% by weight.

27. The method of claim 16 wherein said polypeptide is administered to said subject at a dosage range of about 0.1-50 micrograms.

28. The method of claim 27 wherein said range is about 1-25 micrograms.
